(19) United States
(12) Patent Application Publication
(10) Pub. No.: US 2020/0032208 A1
(43) Pub. Date: Jan. 30, 2020

(54) Methods of Isolating Naive Regulatory T Cells

(71) Applicant: Adaptimmune Ltd, Oxfordshire (GB)

(72) Inventors: Fabian Brunk, Oxfordshire (GB); Jian-Guo Chai, Oxfordshire (GB)

(21) Appl. No.: 16/603,155

(22) PCT Filed: Apr. 4, 2018
(86) PCT No.: PCT/EP2018/058614

§ 371 Date: Oct. 4, 2019

(30) Foreign Application Priority Data
Apr. 5, 2017 (GB) 1705504.7

(51) Int. Cl. C12N 5/0783; A61K 35/17; G01N 33/569

(52) U.S. Cl. CPC C12N 5/0637; A61K 35/17; G01N 33/56966; C12N 2501/998; G01N 2333/70596; G01N 2333/70589; G01N 2333/7051; G01N 2333/70514; C12N 2501/2302

(57) ABSTRACT

This invention relates to the production of isolated naive regulatory T cells by detecting the presence or absence of a panel of surface markers comprising CD3, CD4, CD25, CD45RA, CCR4 and CCR7 on the surface of cells in a population of peripheral blood mononuclear cells (PBMCs); and separating cells on which the presence of the surface markers CD3, CD4, CD25, CD45RA, and CCR7 and the absence of the surface marker CCR4 is detected. Isolated populations of naive regulatory T cells and methods and kits for their production are provided.

### FIELD

This invention relates to methods for the isolation of naïve regulatory T cells.

### BACKGROUND

Regulatory T (Treg) cells are CD4⁺CD25⁺ T cells that control autoimmunity and tolerance (1) through the inhibition of effector T cell responses by a variety of different mechanisms including cell-cell contact and suppressive cytokine production (2).

Autologous regulatory T cells have been used in adoptive immunotherapy to deliver clinical responses in the context of autoimmune diseases. Because the combination of CD4 and CD25 is not sufficient to isolate human regulatory T cells without risk of contamination with potentially autoreactive effector T cells (4), other combinations of markers have been tested. For example, CD4, CD25, and CD127 have been used to isolate populations of human polyclonal CD4⁺CD25⁺CD127⁻ regulatory T cells for use in clinical trials for treating type 1 diabetes (3, 4). However, although CD127 is a useful marker for regulatory T cell isolation in combination with CD4 and CD25, it has limited value in defining subsets of regulatory T cells that are suitable for therapy (3, 4).

Subsets of regulatory T cells have also been identified by inclusion of an additional marker such as CD45RA (5, 6) or CCR4 (7, 8), along with CD4 and CD25. While these additional markers improve subset definition, they do not completely distinguish between regulatory T cell subsets, because not all CD45RA⁺ regulatory T cells are naïve (recently activated conventional CD4⁺ T cells can also be CD45RA⁺). In addition, CD45RA⁺ T cells also contain fractions of CCR4⁺ CD4 T cells which are not naïve T cells.

Similar issues arise when CCR7 is used as an additional marker to isolate defined regulatory T cells subsets (9).

### SUMMARY

The present inventors have identified a defined panel of markers that allows the accurate and consistent isolation of a highly homogeneous subset of naïve regulatory T (Treg) cells that is not contaminated with effector T cells and can be stably expanded in vitro. This subset of naïve regulatory T cells may be useful for example the provision of cells for autologous therapy.

An aspect of the invention provides a method of producing a population of naïve regulatory T cells comprising;
(a) detecting the presence or absence of a panel of surface markers comprising CD3, CD4, CD25, CD45RA, CCR4 and CCR7 on the surface of cells in a population of peripheral blood mononuclear cells (PBMCs); and
(b) separating cells on which the presence of the surface markers CD3, CD4, CD25, CD45RA, and CCR7 and the absence of the surface marker CCR4 is detected to produce an isolated population of naïve regulatory T cells.

The panel of surface markers may further comprise CD45RO and/or CD8.

In some preferred embodiments, the panel consists of the surface markers CD4, CD25, CD45RA, CCR7, CD3, CD45RO, CCR4 and CD8. Cells on which the presence of CD4, CD25, CD45RA, CCR7 and CD3 and the absence of CD45RO, CCR4 and CD8 is detected may be separated from the population.

The isolated population of naïve regulatory T cells may be activated and/or expanded to produce an expanded population of regulatory T cells.

The isolated population of naïve regulatory T cells and/or regulatory T cells expanded therefrom may be modified, stored and/or formulated with a pharmaceutically acceptable excipient.

Another aspect of the invention provides an isolated population of naïve regulatory T cells produced by a method described herein.

Another aspect of the invention provides an expanded population of regulatory T cells produced by expansion of an isolated population of naïve regulatory T cells described herein.

Another aspect of the invention provides a kit for isolating a population of naïve regulatory T cells using a method described herein, the kit comprising;
an antibody that specifically binds to CD3,
an antibody that specifically binds to CD4
an antibody that specifically binds to CD25,
an antibody that specifically binds to CD45RA,
an antibody that specifically binds to CCR4, and;
an antibody that specifically binds to CCR7.

The kit may further comprise one or both of;
an antibody that specifically binds to CD45RO, and;
an antibody that specifically binds to CD8.

The antibodies may be labelled, preferably fluorescently labelled. Preferably, each antibody in the kit is labelled with a different label.

The kit may be suitable for use in fluorescence-activated cell sorting (FACS).

Other aspects and embodiments of the invention are described below.

### BRIEF DESCRIPTION OF FIGURES

FIG. 1 shows the purification of human naïve regulatory T cells from peripheral blood. FIG. 1A shows a flow chart depicting the key steps of one embodiment of the isolation procedure. FIG. 1B shows the validation of Treg identity and naïve phenotype before expansion. FIG. 1C shows the expansion of the purified naïve Treg cells population upon stimulation via TCR and CD28, but in the absence of rapamycin.

FIG. 2 shows that expanded naïve Treg cells maintain hallmark Treg features. FIG. 2A left panel shows representative FACS-plots showing the expression of FoxP3, CTLA-4 and Helios in comparison to T conventional cells (Tconv), cultured in parallel with the Tregs. FIG. 2A right panel shows scatterplots summarizing the percentage of FoxP3/CTLA-4/Helios-positive cells across multiple independent experiments using different donors. FIG. 2B left panel shows representative FACS plots staining for IL-2 in expanded naïve Treg in comparison to Tconvs. FIG. 2B right panel shows a scatterplot summarizing the frequency of cytokine producing cells across multiple independent experiments using different donors.

FIG. 3A shows the suppression of CD8 and CD4 T cell proliferation by expanded Treg cells in response to soluble anti-CD3 and anti-CD28 in vitro. % suppression of CD8 and CD4 T cell responses was determined by following formula: (1-numbers of divisions by T cells in the presence of Treg/numbers of divisions by T cells in the absence of Treg)×100%. Data are representative of three independent experiments in which three different expanded Treg lines were evaluated for suppressive potential against CD8 and CD4 T cells from a common PBLs. FIG. 3B shows examples of suppression of CD8 and CD4 T cell proliferation by Tregs. Upper and lower panels show CD8 and CD4 T cell proliferation in the absence and presence of Tregs respectively.

### DETAILED DESCRIPTION

This invention relates to methods of isolating a subset of naïve regulatory T cells from peripheral blood or extracts thereof. The subset is isolated by detecting the presence or absence of a defined panel of surface markers on the surface of cells within a population of peripheral blood mononuclear cells (PBMCs). The panel may comprise or consist of the positive markers CD3, CD4, CD25, CD45RA, and CCR7, and the negative markers CCR4 and optionally CD45RO and CD8. Cells on which the positive markers in the panel are present and the negative markers in the panel are absent are isolated or sorted from the population of PBMCs to produce an isolated population of naïve regulatory T cells.

Regulatory T cells (or suppressor T cells) are immunosuppressive CD4+ lymphocytes that inhibit effector T cell function. Naïve regulatory T cells are regulatory T cells that have not interacted with a target antigen.

Naïve regulatory T cells isolated as described herein may be non-responsive to TCR- and CD28-mediated stimulation in the absence of IL-2. Upon TCR- and CD28-mediated stimulation in the presence of IL-2, naïve regulatory T cells display proliferative potential and may be cultured to generate an expanded population of regulatory T cells.

Regulatory T cells expanded from the naïve regulatory T cells isolated as described herein may express one, two or all three of FoxP3, CTLA-4 and Helios.

The regulatory T cells may also express suppressive factors, such as CD39 and TIGIT, lymph node homing receptors, such as CD62L, and co-stimulatory molecules, such as CD27 and ICOS. The regulatory T cells may lack expression of T cell exhaustion molecules, such as CD57.

The regulatory T cells may display immunosuppressive activity. For example, the cells may not produce granzyme B and/or inflammatory cytokines, such as IFNγ and IL-2, in response to stimulation (e.g. using PMA and ionomycin) and/or may suppress CD4+ and CD8+ effector T cell proliferation in vitro.

The regulatory T cells may suppress the expression of early activation markers, such as CD40L, CD69 and CD25, in T cells and/or may suppress the activation of B cells by antigen presenting cells. For example, the expression of CD80 on B cells may be reduced in the presence of the expanded regulatory T cells.

Peripheral blood mononuclear cells (PBMCs) are blood cells with round nuclei and include lymphocytes, such as T cells and B cells, and monocytes.

A population of PBMCs suitable for the isolation of naïve regulatory T cells may be obtained from a blood sample from a donor individual. The PBMC population may be isolated or may be present in a sample fraction or sample from the donor individual.

Any suitable donor individual may be used. In some embodiments, the sample may be obtained from a donor individual suffering from a disease condition, for example an autoimmune disease, such as type 1 diabetes, rheumatoid arthritis or psoriasis, or from a healthy individual, for example a healthy individual who is human leukocyte antigen (HLA) matched (either before or after donation) with an individual suffering from such a condition.

PBMCs may be extracted from a blood sample using standard techniques. For example, Ficoll may be used in combination with gradient centrifugation (BÖyum A. Scand J Clin Lab Invest. 1968; 21(Suppl.97): 77-89), to separate whole blood into a top layer of plasma, followed by a layer of PBMCs and a bottom fraction of polymorphonuclear cells and erythrocytes. Suitable reagents are available from commercial suppliers (e.g. (GE Healthcare Bio-Sciences). In some embodiments, the PBMCs may be depleted of CD14+ cells (monocytes).

Naïve regulatory T cells may be isolated as described herein directly from a population of peripheral blood mononuclear cells (PBMCs) obtained from a blood sample or may be extracted or purified from a blood sample.

Preferably, the population of PBMCs is enriched before the naïve regulatory T cells are isolated. For example, the population may be enriched for cells that express CD4 or, more preferably CD25 i.e. the proportion of cells in the population of PBMCs that express CD4 or CD25 may be increased and/or the proportion of cells in the population that do not express CD4 or CD25 may be reduced or depleted in the population of PBMCs to produce an enriched population. The presence or absence of the panel of surface markers may be detected in cells of the enriched population.

Suitable methods for enriching the population for CD4- or more preferably CD25-expressing cells are well known in the art and include magnetic cell sorting (see, for example, Gaudernack et al 1986 J Immunol Methods 90 179; Miltenyi et al Cytometry 11 2 231-238, 1990). For example, the PBMCs may be contacted with magnetic beads coated with anti-CD4 or anti-CD25 antibodies; or the PBMCs may be contacted with biotin conjugated anti-CD4 or anti-CD25 antibodies together with biotin-binding magnetic beads. CD4- or CD25-expressing cells in the population bind to the magnetic beads and may be separated from other cells in the population using a magnetic field to produce the enriched population.

The naïve regulatory T cells are isolated from the population of PBMCs according to the presence or absence of the panel of surface markers on the cell surface. The panel may comprise or consist of one of the following combinations of surface markers;

### (i) CD3, CD4, CD25, CD45RA, CCR4, CCR7

### (ii) CD3, CD4, CD25, CD45RA, CCR4, CCR7, CD45RO

(iii) CD3, CD4, CD25, CD45RA, CCR4, CCR7, CD8

### (iv) CD3, CD4, CD25, CD45RA, CCR4, CCR7, CD8 and CD45RO

Some surface markers in the panel, such as CD4, CD25, CD45RA, CCR7 and CD3, may be positive markers whose presence on the cell surface is used to identify and isolate naïve regulatory T cells. In methods described herein, the presence of one or more positive markers may be detected on cells in the PBMC population.

CD4 (Gene ID 920) is a membrane glycoprotein that is expressed in T cells, B cells macrophages, and granulocytes. Human CD4 may have the reference amino acid sequence NP_000607.1 and the reference nucleic acid sequence NM_000616.4.

CD25 (Gene ID 3559; also called IL-2RA) is the alpha chain of the interleukin 2 receptor (IL-2RA). Human CD25 may have the reference amino acid sequence NP_000408.1 and the reference nucleic acid sequence NM_000417.2. Naïve regulatory T cells may be CD25high and may be distinguished from T cells that are CD25intermediate-high by standard immunofluorescent techniques.

CD45RA is an isoform of CD45 (Gene ID 5788; also called protein tyrosine phosphatase; PTPRC). Human CD45RA may have the reference amino acid sequence NP_002829.3 and the reference nucleic acid sequence NM_002838.4

CCR7 (Gene ID 1236) is a G protein-coupled chemokine receptor. Human CCR7 may have the reference amino acid sequence NP_001288643.1 and the reference nucleic acid sequence NM_001301714.1. Naïve regulatory T cells may be CCR7intermediate-high or CCR7high. Populations of naïve regulatory T cells may have a narrow CCR7 expression spectrum ranging from CCR7intermediate-high to CCR7high and may be distinguished from other T cell populations which have a broad CCR7 expression spectrum ranging from CCR7neg to CCR7high.

CD3 associates with the T cell receptor (TCR) to form the TCR complex on the surface of T cells. CD3 may be CD3delta, CD3gamma, CD3 epsilon or CD3 zeta. Human CD3 gamma (Gene ID 917) may have the reference amino acid sequence NP_000064.1 and the reference nucleic acid sequence NM_000073.1. Human CD3delta (Gene ID 915) may have the reference amino acid sequence NP_000723.1 and the reference nucleic acid sequence NM_000732.4. Human CD3epsilon (Gene ID 916) may have the reference amino acid sequence NP_000724.1 and the reference nucleic acid sequence NM_000733.3. CD3zeta (Gene ID 919) may have the reference amino acid sequence NP_000725.1 and the reference nucleic acid sequence NM_000734.3.

Other surface markers in the panel, such as CD45RO, CCR4 and CD8, may be negative markers whose absence from the cell surface is used to identify and isolate naïve regulatory T cells. In methods described herein, the absence of one or more negative markers may be detected on cells in the PBMC population

CD45RO is the shortest isoform of CD45 (Gene ID 5788; also called protein tyrosine phosphatase; PTPRC) and lacks the RA, RB, and RC exons. Human CD45RO may have the reference amino acid sequence NP_563578.2 and the reference nucleic acid sequence NM_080921.3

CCR4 (Gene ID: 1233) is a G-protein-coupled chemokine receptor. Human CCR4 may have the reference amino acid sequence NP_005499.1 and the reference nucleic acid sequence NM_005508.1. Naïve regulatory T cells may be CCR4 negative or CCR4low. CD8 (Gene ID: 925) is a glycoprotein found on the surface of cytotoxic T lymphocytes. Human CD8a (Gene ID: 925) may have the reference amino acid sequence NP_001139345.1 and the reference nucleic acid sequence NM_001145873.1. Human CD8b (Gene ID: 926) may have the reference amino acid sequence NP_001171571.1 and the reference nucleic acid sequence NM_001178100.1.

In some preferred embodiments, the panel of surface markers may comprise CD4, CD25, CD45RA, CCR7, CD3, CD45RO, CCR4 and CD8.

Cells in the PBMC population on whose surface the presence of CD3, CD4, CD25, CD45RA, and CCR7 and the absence of CCR4 is detected may be separated from the population (i.e. CD3⁺, CD4⁺, CD25⁺, CD45RA⁺, CCR4⁻, CCR7⁺ cells may be separated or sorted from the other cells in the PBMC population).

A method of producing a population of naïve regulatory T cells may comprise;
(i) providing a population of peripheral blood mononuclear cells (PBMCs); and
(ii) isolating cells that express the surface markers CD3, CD4, CD25, CD45RA and CCR7 and do not express the surface markerCCR4 from the population of PBMCs, thereby producing an isolated population of naïve regulatory T cells.

Preferably, cells in the PBMC population on whose surface the presence of CD3, CD4, CD25, CD45RA, and CCR7 and the absence of CD45RO, and CCR4 and optionally CD8, is detected are separated from the population (i.e. the subset of CD3⁺, CD4⁺, CD25⁺, CD45RA⁺, CD45RO⁻, CCR4⁻, CCR7⁺ cells are separated or sorted from the other cells in the PBMC population).

In some embodiments, a surface marker may be detected as present when the expression of the marker on the cell is high or intermediate-high expression, as determined relative to reference markers using standard immunofluorescent techniques, such as FACS with default parameters. A surface marker may be detected as absent when the expression of the marker on the cell is low or negative.

In some embodiments, cells may be isolated from the PBMC population on the basis of the amount of expression of one or more surface markers in the panel. For example, CD25high, CCR7intermediate high/high and/or CCR4neg/low cells may be isolated from the population.

In addition to determining the presence or absence of the panel of surface markers on the surface of cells, a method may further comprise determining the viability of cells in the population of PMBCs. The presence of the positive markers in the panel, the absence of the negative markers in the panel and the presence of viability may be together indicative of a cell to be separated or sorted from the population of PBMCs.

The viability of cells in the population of PBMCs may be determined using conventional techniques. For example the cells may stained with a viability dye. Suitable dyes are well known in the art and include dyes which selectively stain dead cells, such as Fixable Viability Dye eFluor® 350, 405, 488, 633 or 780, 7-amino-actinomycin D (7-AAD) and propidium iodide (Thermo Fisher Scientific Inc) and dyes which selectively stain live cells, such as calcein AM and calcein violet AM (Thermo Fisher Scientific Inc).

The presence of a dead-cell binding viability dye or the absence of live-cell binding viability dye is indicative that a cell is dead and should not be separated from the PBMC population. The absence of a dead-cell binding viability dye or the presence of live-cell binding viability dye is indicative that a cell is viable and may be separated from the PBMC population subject to the presence or absence of the panel of surface markers.

The presence or absence of the panel of surface markers on cells in the population of PBMCs may be detected by any suitable technique. A range of suitable techniques are available in the art, including immunocytochemistry, immunofluorescence, immunoblotting, magnetic activated cell sorting (MACS) and flow cytometry techniques such as fluorescence activated cell sorting (FACS) (see for example Reinherz et al (1979) PNAS 76 4061).

For example, the presence or absence of the panel of surface markers may be detected by;
contacting the population of PBMCs with antibodies which bind specifically to the surface markers in the panel, such that each surface marker in the panel binds to a different antibody and;
separating cells from the population according to the combination of antibodies that are bound to the surface of each cell.

The PBMCs may be contacted with antibodies which bind to the positive markers in the panel. Cells which have antibodies to the positive markers bound to their surface may be isolated from the PBMC population. The PBMCs may also be contacted with antibodies which bind to the negative markers in the panel. Cells which do not have antibodies to the negative markers bound to their surface may be isolated from the PBMC population.

For example, for a panel of markers consisting of CD4, CD25, CD45RA, CCR7, CD3, CD45RO, CCR4 and CD8, the PBMCs may be contacted with anti-CD3, anti-CD4, anti-CD25, anti-CD45RA, anti-CCR7, anti-CD45RO, anti-CCR4, and anti-CD8 antibodies. Cells which have anti-CD4, anti-CD25, anti-CD45RA, anti-CD3 and anti-CCR7 antibodies bound to their surface and which do not have anti-CD45RO, anti-CCR4, and anti-CD8 antibodies bound to their surface (i.e. CD3+, CD4+, CD8−, CD25+, CD45RA+, CD45RO−, CCR4− and CCR7+ cells) may be sorted away from the PBMC population.

The PBMCs may be contacted with all of the antibodies to the markers in the panel sequentially or more preferably simultaneously. For example, the PBMCs may be contacted with a mixture which comprises antibodies to all of the markers in the panel.

The antibodies may be fluorescently labelled, for example for separation by FACS. Preferably, the antibodies are conjugated to fluorophores before contact with the population of PBMCs. Antibodies to different markers in the panel may be conjugated to different fluorophores to allow the presence or absence of the individual markers in the panel to be determined. Suitable fluorophores are well-known in the art and include peridinin chlorophyll, phycoerythrin and allophycocyanin. Fluorescently labelled antibodies which bind to the surface markers in the panel may be produced using standard techniques or obtained from commercial suppliers (e.g. Abcam Ltd, Cambridge UK; BD Pharmingen, Heidelberg, Germany).

Preferably, the presence or absence of the panel of surface markers is determined and the cells separated from the population of PBMCs in a single step, for example using a flow-cytometry technique, such as fluorescence-activated cell sorting (FACS). Suitable techniques and equipment for separating cells using FACS are well-known in the art (e.g. BD FACSAria II or BD FACSMelody; BD Biosciences).

The population of naïve regulatory T cells separated from the PBMCs using the panel of markers may comprise at least 80%, at least 90%, at least 95%, at least 98% or at least 99% naive regulatory T cells.

The initial population of naïve regulatory T cells isolated from the population of PBMCs may be cultured in vitro to produce an expanded population of regulatory T cells.

Suitable methods for expanding regulatory T cells are known in the art (2). In some embodiments, the isolated naïve regulatory T cells may be exposed to a T cell receptor (TCR) agonist in the presence of IL2. Suitable TCR agonists include ligands, such as a peptide displayed on a class I or II MHC molecule on the surface of an antigen presenting cell, such as a dendritic cell, and soluble factors, such as anti-TCR antibodies.

An anti-TCR antibody may specifically bind to a component of the TCR-CD3 complex, such as TCR-α, TCR-β, εCD3, δCD3 or γCD3. Anti-TCR antibodies suitable for TCR stimulation are well-known in the art (e.g. anti-εCD3 mAb OKT3) and available from commercial suppliers (e.g. Abcam Ltd, Cambridge, UK; eBioscience CO USA).

In some embodiments, naïve regulatory T cells may be activated by exposure to anti-αCD3 antibodies and IL2, more preferably, by exposure to anti-αCD3 antibodies, anti-αCD28 antibodies and IL2. For example, the naïve regulatory T cells may be activated with anti-CD3 and anti-CD28 antibody coated beads and IL2. For example, naïve regulatory T cells may be activated, without feeder cells (antigen presenting cells) or antigen, using antibody coated beads, for example magnetic beads coated with anti-CD3 and anti-CD28 antibodies, such as Dynabeads® Human T-Activator CD3/CD28 (ThermoFisher Scientific) and IL2.

Preferably, the isolated naïve regulatory T cells are activated and expanded in the absence of an mTOR inhibitor, such as rapamycin. For example, the naïve regulatory T cells may expanded using anti-CD3 and anti-CD28 antibody coated beads and IL2 in the absence of rapamycin.

Expression of one or more markers in the panel may be altered during expansion. For example, expression of CD45RA may be reduced or lost during expansion (e.g. from CD45RA+ to CD45RA−); expression of CCR4 may be increased (e.g. from CCR4− to CCR4+); and/or expression of CD45RO may be increased (e.g. from CD45RO− to CD45RO+). In some embodiments, CCR4 may be absent from the naïve regulatory T cells of the isolated population and present on the regulatory T cells of the expanded population. Similarly, CD45RA may be present from the naïve regulatory T cells of the isolated population and absent on the regulatory T cells of the expanded population. In addition, the expression of CD4 and CD25 may be increased following expansion.

In some embodiments, the regulatory T cells may adopt a memory or effector phenotype after expansion.

The population may be expanded 100 fold or more, 500 fold or more, 1000 fold or more or 2000 or more or 5000 fold or more. For example, the population of isolated naïve regulatory T cells may be expanded 1,000-4,000 fold to produce the expanded population.

In some preferred embodiments, the isolated population may be expanded to a therapeutic dose of regulatory T cells. For example, the population may be expanded to contain 10⁷ to 10⁹ regulatory T cells.

The naïve regulatory T cells may be cultured using any convenient technique to produce the expanded population.

The naïve regulatory T cells may be cultured as described herein in any suitable system, including stirred tank fermenters, airlift fermenters, roller bottles, culture bags or dishes, and other bioreactors, in particular hollow fibre bioreactors. The use of such systems is well-known in the art.

Numerous culture media suitable for use in the proliferation of naïve regulatory T cells ex vivo are available, in particular complete media, such as AIM-V, Iscoves medium, RPMI-1640 (Invitrogen-GIBCO) or X-VIVO 10 or X-VIVO 15 medium (Lonza). Preferred media may be supplemented with high dose (300 U/ml) IL-2 and further may be supplemented with other factors such as serum, serum proteins and selective agents. For example, in some embodiments, RPMI-1640 medium containing 2 mM glutamine, 10% FBS, 25 mM HEPES, pH 7.2, 1% penicillin-streptomycin, and 55 μM β-mercaptoethanol and optionally supplemented with 20 ng/ml recombinant IL-2 may be employed. The culture medium may be supplemented with the agonistic or antagonist factors described above at standard concentrations which may readily be determined by the skilled person by routine experimentation.

Conveniently, cells are cultured at 37° C. in a humidified atmosphere containing 5% CO₂ in a suitable culture medium.

Methods and techniques for the culture of naïve regulatory T cells and other mammalian haematopoietic cells are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52)

The expanded population of regulatory T cells may comprise at least 80%, at least 90%, at least 95%, at least 98% or at least 99% regulatory T cells.

Following the isolation and expansion of the initial population, the regulatory T cells may be modified, for example, to be specific for or recognise a target antigen. For example, the naïve regulatory T cells may be modified to express a heterologous antigen receptor such as a chimeric antigen receptor, T body receptor or heterologous αβTCR heterodimer. The heterologous receptor may be specific for an antigen. Heterologous receptors suitable for expression in naïve regulatory T cells may have a known specificity and avidity for a selected target antigen.

Naïve regulatory T cells or expanded regulatory T cells may be genetically modified to express a heterologous antigen receptor using any convenient technique. For example, a heterologous nucleic acid, such as a nucleic acid construct or vector encoding the heterologous receptor may be introduced into the cells in the culture medium. This may be useful in altering the function or antigenic specificity of the naïve regulatory T cells, for example, by causing the naïve regulatory T cells to express a heterologous antigen receptor. For example, a construct encoding a heterologous antigen receptor such as a TCR or TCR subunit which is specific for a particular antigen, for example a disease-associated antigen, or a construct encoding a dominant negative form of a receptor, such as TGFβ receptor II, may be introduced into the cells. The genetic modification of naïve regulatory T cells to express heterologous antigen receptors and the subsequent use of such genetically modified T-cells in adoptive T-cell therapy are well known in the art.

When introducing or incorporating a heterologous nucleic acid into a cell, certain considerations must be taken into account, well known to those skilled in the art. The nucleic acid to be inserted should be assembled within a construct or vector which contains effective regulatory elements which will drive transcription in the target cell. Suitable techniques for transporting the constructor vector into the cell are well known in the art and include calcium phosphate transfection, DEAE-Dextran, electroporation, liposome-mediated transfection and transduction using retrovirus or other virus, e.g. vaccinia or lentivirus. For example, solid-phase transduction may be performed without selection by culture on retronectin-coated, retroviral vector-preloaded tissue culture plates.

Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992.

Optionally, after isolation, expansion and optional modification, the population of regulatory T cells may be stored, for example by lyophilisation and/or cryopreservation, before use.

The naïve regulatory T cells or expanded regulatory T cells may be formulated with a pharmaceutically acceptable excipient to produce a pharmaceutical composition.

Pharmaceutical compositions comprising naïve regulatory T cells suitable for administration (e.g. by infusion) may include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

In some preferred embodiments, the naïve regulatory T cells or expanded regulatory T cells may be formulated into a pharmaceutical composition suitable for intravenous infusion into an individual. For example, the naïve regulatory T cells or expanded regulatory T cells may be formulated into a sterile infusion solution comprising a plasma electrolyte formulation (e.g. plasma-lyte; Rizoli et al J. Trauma. 2011 May; 70 (5 Suppl): 517-8), dextrose, NaCl and human serum albumin (HSA).

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Another aspect of the invention provides a pharmaceutical composition comprising a population of expanded regulatory T cells as described herein.

Regulatory T cells produced and expanded as described above may be administered to a recipient individual, for example for the treatment of an immune condition, such as an autoimmune disease.

Another aspect of the invention provides a method of treatment of an immune condition may comprise;
administering a population of regulatory T cells produced by method described herein to an individual in need thereof.

Another aspect of the invention provides a population of regulatory T cells produced by method described herein for use in method of treatment of an immune condition in an individual.

Another aspect of the invention provides the use of a population of regulatory T cells produced by method described herein in the manufacture of a medicament for use in a method of treatment of an immune condition in an individual.

Immune conditions may include autoimmune diseases, such as celiac disease, type 1 diabetes, Grave's disease, inflammatory bowel disease, multiple sclerosis, psoriasis, rheumatoid arthritis, and systemic lupus erythematosus. Immune conditions may also include allergy, inflammation, sepsis, and acute and chronic Graft versus host disease (GvHD)

The expanded population of regulatory T cells may be administered intravenously, for example by infusion into the individual.

The expanded population of regulatory T cells may be autologous i.e. the naïve regulatory T cells were originally obtained from the same individual to whom they are subsequently administered (i.e. the donor and recipient individual are the same). A suitable expanded population of regulatory T cells for administration to a recipient individual may be produced by a method comprising providing an initial population of PBMCs obtained from the individual, isolating a population of naïve regulatory T cells from the PBMCs as described above and culturing the isolated naïve regulatory T cells to produce an expanded population.

The expanded population of regulatory T cells may be allogeneic i.e. the naïve regulatory T cells were originally obtained from a different individual to the individual to whom they are subsequently administered (i.e. the donor and recipient individual are different). The donor and recipient individuals may be HLA matched to avoid GVHD and other undesirable immune effects. A suitable expanded population of regulatory T cells for administration to a recipient individual may be produced by a method comprising providing an initial population of PBMCs obtained from a donor individual, isolating a population of naïve regulatory T cells from the PBMCs as described above and culturing the isolated naïve regulatory T cells to produce an expanded population.

Following administration of the regulatory T cells, T cell mediated immune responses in the recipient individual may be reduced or suppressed. This may have a beneficial effect in the individual, for example in treating an autoimmune disease or other immune condition.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, an individual susceptible to or at risk of the occurrence or re-occurrence of cancer may be treated as described herein. Such treatment may prevent or delay the occurrence or re-occurrence of cancer in the individual.

The regulatory T cells or the pharmaceutical composition comprising the regulatory T cells may be administered to a subject by any convenient route of administration, whether systemically/peripherally or at the site of desired action, including but not limited to; parenteral, for example, by infusion. Infusion involves the administration of the T cells in a suitable composition through a needle or catheter. Typically, T cells are infused intravenously or subcutaneously, although the T cells may be infused via other non-oral routes, such as intramuscular injections and epidural routes. Suitable infusion techniques are known in the art and commonly used in therapy (see, e.g., Rosenberg et al., New Eng. J. of Med., 319:1676, 1988).

Typically, the number of cells administered is from about 10⁵ to about 10¹⁰ per Kg body weight, typically 10⁸-10¹⁰ cells per individual, typically over the course of 30 minutes, with treatment repeated as necessary, for example at intervals of days to weeks. It will be appreciated that appropriate dosages of the expanded regulatory T cells, and compositions comprising the naïve regulatory T cells, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

The expanded population of regulatory T cells may be administered in combination with one or more other therapies, such as cytokines e.g. IL-2.

In some preferred embodiments, the administration of IL-2, for example at low doses such as <3×10⁶ IU/m²/day, may be useful in promoting regulatory T cell survival in vivo.

The one or more other therapies may be administered by any convenient means, preferably at a site which is separate from the site of administration of the naïve regulatory T cells.

Administration of the expanded regulatory T cells can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. Preferably, the regulatory T cells are administered in a single transfusion of a least 1×10⁹ T-cells.

Another aspect of the invention provides a kit for producing a population of naïve regulatory T cells comprising;
a fluorescently labelled antibody that specifically binds to CD3,
a fluorescently labelled antibody that specifically binds to CD4,
a fluorescently labelled antibody that specifically binds to CD25,
a fluorescently labelled antibody that specifically binds to CCR4,
a fluorescently labelled antibody that specifically binds to CD45RA, and;
a fluorescently labelled antibody that specifically binds to CCR7.

The kit may further comprise;
a fluorescently labelled antibody that specifically binds to CD45RO, and/or
a fluorescently labelled antibody that specifically binds to CD8.

The kit may be suitable for sorting naïve regulatory T cells from a population of PBMCs, for example by FACS, as described herein.

Suitable fluorescently labelled antibodies are readily available in the art.

A kit for use in a method described herein may include one or more articles and/or reagents for performance of the method, such as means for isolating the PBMCs from blood, purification columns, sample handling containers (such components generally being sterile), and other reagents required for the method, such as buffer solutions, activation reagents such as anti-CD3 and anti-CD28 coated beads, IL2, red blood cell lysis reagent, culture media, and other reagents.

The kit may include instructions for use in a method of isolating naïve regulatory T cells as described above.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Certain aspects and embodiments of the invention will now be illustrated by way of example and with reference to the figures described above.

### Experiments

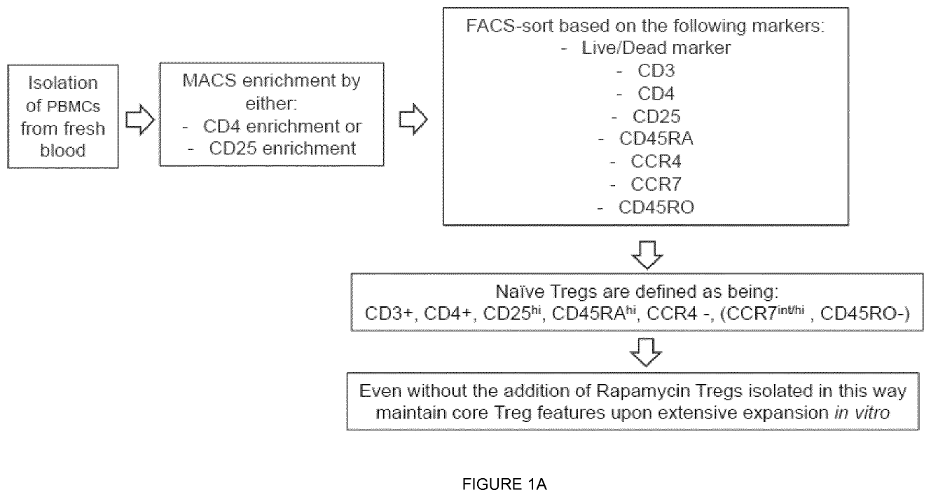

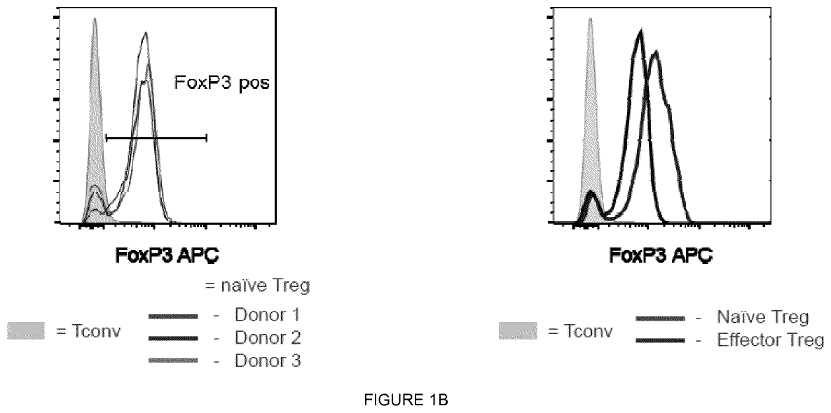

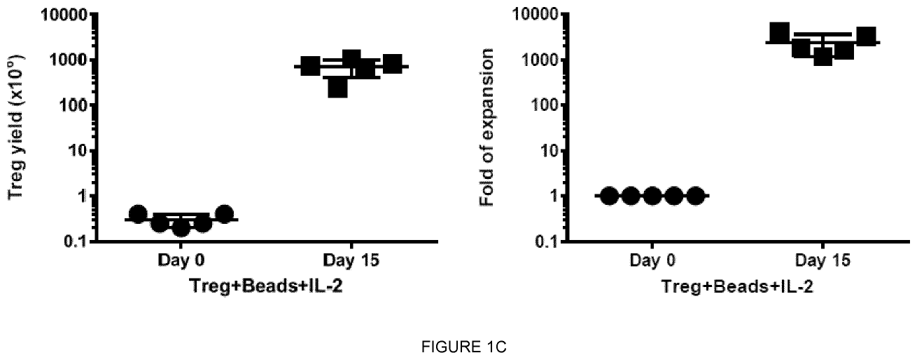

We have designed a novel panel of markers by which naïve human regulatory T cells of high purity can be readily isolated from peripheral blood. FIG. 1A shows an overview of the workflow of this process (FIG. 1A).

Peripheral blood mononuclear cells (PBMCs) were isolated using standard density gradient techniques. Magnetic activated cell sorting (MACS)-based enrichment of either CD4−, and/or CD25 positive cells was used to pre-enrich for regulatory T cells (Tregs). An initial pre-enrichment stage for CD25+ PBMC was performed prior to cell sorting. In brief, PBMCs were incubated with either anti-human CD25 magnetic beads (MicroBeads™, Miltenyi Biotec) or biotin-conjugated anti-CD25 followed by anti-biotin magnetic beads. After separation, the frequency of CD25 expressing cells in the positive fraction was increased at least 10-fold. The pre-enriched cell suspension was stained with an antibody-cocktail to CD3, CD4, CD25, CD45RA, CCR4, CCR7 and CD45RO as well as a dead cell exclusion dye. Naïve Tregs were defined as being (live, single cells) CD3+, CD4+, CD25^{high}, CD45RA^{high}, CCR4^{neg}, CCR7^{int/high} CD45RO^{neg}. The CD25 enriched population was stained with dead cell exclusion dye and conjugated antibodies to CD4, CD3, CD8, CD25, CD45RA, CD45RO, CCR4 and CCR7 before running on a cell sorter.

Based on this panel, naïve Treg cells displaying a phenotype of CD3⁺CD4⁺CD8⁻CD25⁺CD45RA⁺CD45RO⁻CCR4⁻CCR7⁺ were precisely defined and sorted with a purity of >95-99%. This high sorting purity makes the use of Rapamycin, which is routinely used to prevent the outgrowth of contaminating conventional T cells, obsolete and it was omitted. Rapamycin can also delay or impair Treg activation and proliferation, so the absence of rapamycin is beneficial for the rapid expansion of Tregs to therapeutic doses.

The regulatory T cell identity of the isolated population was validated using a staining panel that included the Treg lineage marker FoxP3. Conventional T cells were gated as negative control. Unlike the non-regulatory, conventional CD4 T cells (Tconv), freshly isolated naïve Treg cells isolated from three donors showed robust expression of FOXP3 (>90%; FIG. 1B). This shows that naïve Tregs gated using the marker panel are indeed bona fide Tregs. While both naïve- and effector Tregs are CD25^{high} and FoxP3^{positive}, effector Tregs are known to express higher levels of FoxP3 and CD25 as compared to naïve Tregs.

Sorted naïve Tregs expressed a lower level of FOXP3 (and CD25, data not shown) compared with effector Treg cells purified from the same donor (being CD3⁺CD4⁺CD8⁻CD25+CD127^{dim}CD45RA⁻CCR4⁺) (FIG. 1B) (3).

Naïve Treg cells seems to be the only Treg subset that is highly expandable in vitro under current expansion protocols using anti-CD3 and anti-CD28 beads together with IL-2 (4). Upon stimulation via TCR and CD28, but in the absence of rapamycin, naïve Treg cells purified using our Treg panel from the peripheral blood of several individual donors were capable of undergoing 1,000-4,000 fold expansion over 14-15 days (FIG. 1C).

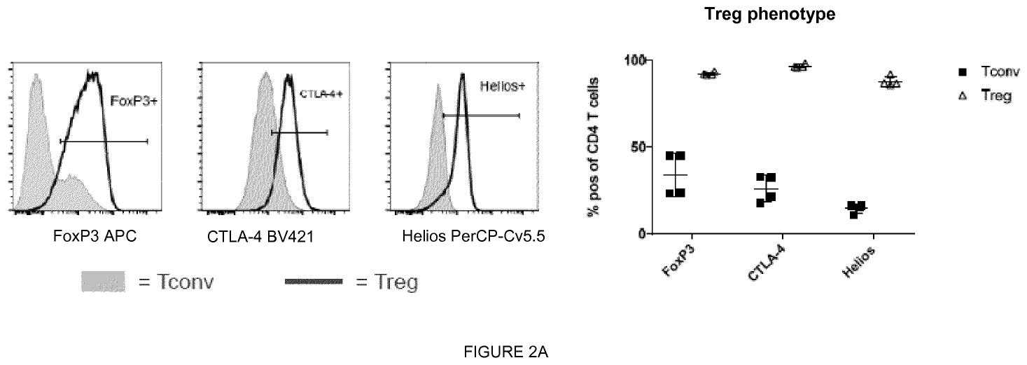

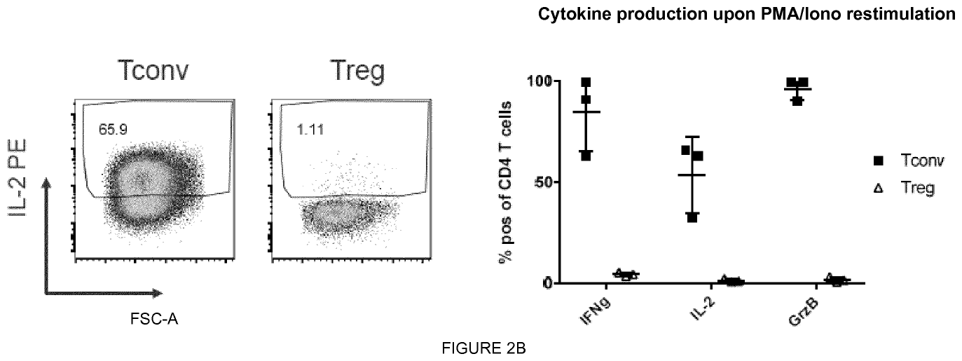

Expanded Treg cells, but not expanded conventional CD4 T cells (Tconv) from the same donors, expressed high levels of FOXP3, CTLA4 and Helios upon extensive expansion in vitro, even in the absence of rapamycin (FIG. 2a).

Importantly, expanded Tregs upregulated molecules closely associated with Treg suppressive activity (CD39 and TIGIT) whilst maintaining expression of lymph node homing receptors (CD62L and CCR7) and co-stimulatory molecules (CD27 and ICOS), but did not express the T cell exhaustion molecule CD57.

Tregs expanded in vitro often acquire features of Tconvs, including pro-inflammatory cytokine production, either by losing their Treg identity or due to outgrowth of contaminating Tconvs. To rule out these possibilities, we re-stimulated expanded naïve Treg with Cell Stimulation Cocktail® (eBioscience, #00-4975-93)+protein transport inhibitors for 4-5 h, or with protein transport inhibitors alone as control. Cytokine production was then assessed by intracellular FACS staining using standard protocols. Tconvs, cultured alongside Tregs were used as positive control. The expanded Tregs did not produce pro-inflammatory cytokines upon re-stimulation. Comparing cytokine profiles (induced following PMA/lonomycin stimulation in the presence of transport inhibitors) between expanded Tconv and expanded Treg cells, we found only Tconv cells contained very high frequencies of IFNg-, Granzyme B (GrzB)- and IL-2-producing T cells. In sharp contrast, the frequency of cytokine production amongst expanded Treg cells was extremely low, often at background level (FIG. 2B). Collectively, these phenotypic analyses demonstrate that expanded naïve Tregs represent a stable population which retains the original lineage signature even after extensive in vitro expansion.

Treg suppressive activity was determined using a standard in vitro functional assay whereby suppression of the proliferation of conventional CD4 and CD8 T cells activated by anti-CD3 and anti-CD28 mAbs was measured.

Expanded naïve Treg cultures from 3 donors were used as suppressors in Treg suppression assays. Frozen PBLs (CD14-depleted PBMC containing 40% CD4 and 20% CD8 T cells) from a common health donor were selected as standard responders, allowing the direct comparison of regulatory efficacy by the different expanded Treg lines. After thawing, the PBLs were rested in the culture media (Ex-vivo 15 supplemented with 10% AB sera and antibiotic) for 1 day, before labelling with Cell-Trace Violet dye (CTV), which was performed according to manufacturer's instructions (Life Science). CTV-labelled PBLs were utilised as responders and stimulated with soluble anti-CD3 and anti-CD28 mAb (the final concentration is 2 ug/ml) in the absence or presence of expanded Treg cells which were rested for one day before assay in 96 round-bottomed plates. Additional controls included Treg stimulated with anti-CD3 and anti-CD28 and responders culture with media alone. The number of the PBLs was fixed as 5×10⁴/well while the number of Treg were titred, resulting in different ratios between the PBLs: Treg (1: 0 (no Treg added), 1:1/2, 1:1/4, 1:1/8, 1:1/16, 1:1/32, and 1:1/64). After 5 days of culture, the individual wells under the same culture conditions were pooled, stained with Live-Dead dye (Aqua or eF780), anti-CD4 and anti-CD8 before running on a MACSQuant. Gated CD8 and CD4 responder T cells were further analysed for the dilution of CTV-dye. Because CD4 responder T cells were labelled with CTV whereas Treg cells were not, these two CD4 populations could be separated.

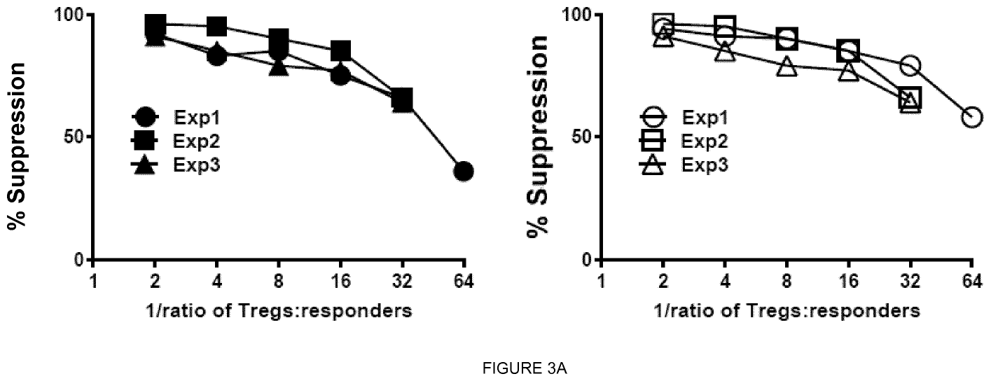

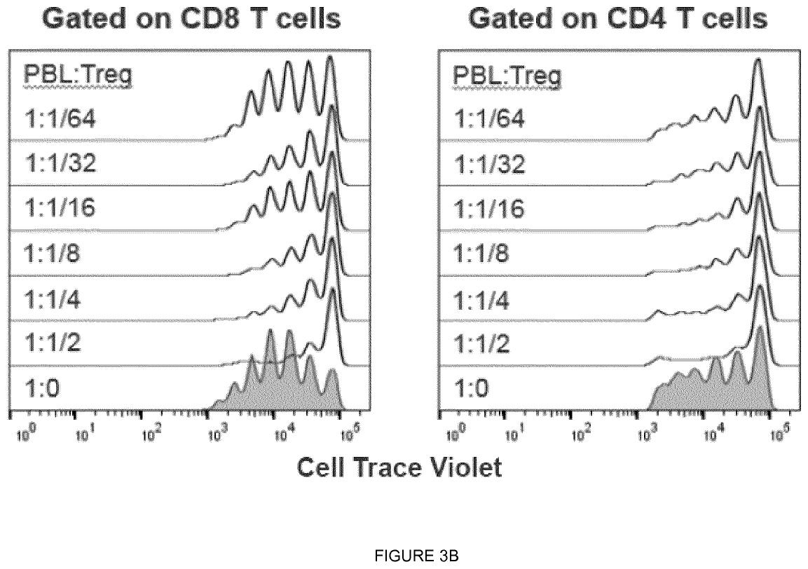

Expanded naïve Treg cultures from 3 donors were found to be capable of inhibiting >80% CD4 and CD8 T cell proliferation even in the presence of lower numbers of Treg in the co-cultures (FIGS. 3A, 3B).

In summary, the Treg panel described herein facilitates isolation of the naïve Treg subset with high purity. As a result, inclusion of rapamycin during their subsequent expansion is not required allowing robust Treg expansion achieving cell numbers required for therapeutic use. Importantly, expanded Tregs function as powerful suppressors, display Treg lineage hallmarks and maintaining expression of key co-stimulatory as well as essential homing receptors.

### REFERENCES

1. Sakaguchi S, et al J Immunol. 1995; 155:1151-1164
2. Tang Q et al Nat Immunol. 2008; 9:239-244
3. Marek-Trzonkowska N et al Clin Immunol. 2014 July; 153(1):23-30.
4. Bluestone J A et al Sci Transl Med. 2015 Nov. 25; 7(315):315ra189
5. Miyara M, et al Immunity. 2009 Jun. 19; 30(6):899-911.
6. Hoffmann P et al Blood. 2006 Dec. 15; 108(13):4260-7.
7. Baatar D et al J Immunol. 2007 Apr. 15; 178(8):4891-900.
8. Sugiyama D et al Proc Natl Acad Sci USA. 2013 Oct. 29; 110(44):17945-50
9. Valmori D et al J Clin Invest. 2005 July; 115(7):1953-62.

1. A method of producing a population of naïve regulatory T cells comprising;
(a) detecting the presence or absence of a panel of surface markers comprising CD3, CD4, CD25, CD45RA, CCR7, and CCR4 on the surface of cells in a population of peripheral blood mononuclear cells (PBMCs); and
(b) separating from the PBMC population cells on which the presence of the surface markers CD3, CD4, CD25, CD45RA, and CCR7 and the absence of the surface marker CCR4 is detected to produce an isolated population of naïve regulatory T cells.

2. A method according to claim 1 wherein the panel further comprises CD45RO and cells on which the absence of CD45RO is detected are separated from the population of PBMCs.

3. A method according to any one of the preceding claims wherein the panel further comprises CD8 and cells on which the absence of CD8 is detected are separated from the population of PBMCs.

4. A method according to any one of the preceding claims wherein the panel consists of the surface markers CD4, CD25, CD45RA, CCR7, CD3, CD45RO, CCR4 and CD8 and cells on which the presence of CD4, CD25, CD45RA, CCR7 and CD3 and the absence of CD45RO, CCR4 and CD8 is detected are separated from the population of PBMCs.

5. A method according to any one of the preceding claims wherein the population of PBMCs is contacted with a viability dye and cells which are detected as viable are separated from the population.

6. A method according to any one of the preceding claims wherein presence or absence of the panel of surface markers is detected by contacting the population of PBMCs with antibodies which bind specifically to the surface markers in the panel, such that each surface marker in the panel binds to a different antibody.

7. A method according to claim 6 wherein cells are separated from the population according to the combination of antibodies that are bound to the surface of each cell.

8. A method according to any one of the preceding claims wherein the cells are separated from the population by flow cytometry.

9. A method according to any one of the preceding claims wherein the cells are separated from the population by fluorescent activated cell sorting.

10. A method according to any one of the preceding claims wherein the population of PBMCs is enriched for CD4-expressing cells or CD25-expressing cells

11. A method according to any one of the preceding claims wherein the population of PBMCs is extracted from a blood sample obtained from a donor individual.

12. A method according to any one of the preceding claims wherein the population of PBMCs is present in a blood sample obtained from a donor individual

13. A method according to any one of the preceding claims comprising expanding the population of naïve regulatory T cells.

14. A method according to claim 13 wherein the population of naïve regulatory T cells is expanded in the absence of rapamycin.

15. A method according to claim 13 or claim 14 wherein the population of naïve regulatory T cells is expanded in the presence of anti-CD3 antibodies, anti-CD28 antibodies and IL2.

16. A method according to any one of claims 13 to 15 wherein the expanded regulatory T cells express FoxP3, CTLA-4 and Helios.

17. A method according to any one of claims 13 to 16 wherein the expanded regulatory T cells suppress CD4+ and CD8+ effector T cell function.

18. A method according to any one of claims 13 to 17 comprising modifying the expanded population of regulatory T cells.

19. A method according to claim 18 comprising modifying the expanded population of regulatory T cells to express a heterologous antigen receptor.

20. A method according to any one of claims 13 to 19 comprising storing the expanded population of regulatory T cells.

21. A method according to any one of claims 13 to 20 comprising formulating the expanded population of regulatory T cells with a pharmaceutically acceptable excipient.

22. A method according to any one of claims 13 to 21 comprising administering the expanded regulatory T cells to a recipient individual.

23. A method according to claim 22 wherein the recipient individual has an immune condition.

24. A method according to claim 22 or claim 23 wherein the donor individual and the recipient individual are the same.

25. A method according to claim 22 or claim 23 wherein the donor individual and the recipient individual are different.

26. An isolated population of naïve regulatory T cells produced by a method according to any one of claims 1 to 12.

27. An isolated population according to claim 26 wherein at least 95% of the cells in the population are CD3⁺, CD4⁺, CD8⁻, CD25⁺, CD45RA⁺, CD45RO⁻, CCR4⁻, CCR7⁺ cells.

28. An expanded population of regulatory T cells produced by a method according to any one of claims 13 to 21.

29. A population of regulatory T cells according to any one of claims 26 to 28 for use in a method of treatment of the human or animal body.

30. A method of treatment of an immune condition comprising;
administering a population of regulatory T cells according to any one of claims 26 to 28 to an individual in need thereof.

31. A population of regulatory T cells according to any one of claims 26 to 28 for use in method of treatment of an immune condition in an individual.

32. Use of a population of regulatory T cells according to any one of claims 26 to 28 in the manufacture of a medicament for use in a method of treatment of an immune condition in an individual.

33. A method according to claim 30, a population for use according to claim 31 or a use according to claim 32 wherein the immune condition is an autoimmune disease.

34. A kit for isolating a population of naïve regulatory T cells comprising;
an antibody that specifically binds to CD3,
an antibody that specifically binds to CD4,
an antibody that specifically binds to CD25,
an antibody that specifically binds to CD45RA,
an antibody that specifically binds to CCR4, and;
an antibody that specifically binds to CCR7.

35. A kit according to claim 34 further comprising
an antibody that specifically binds to CD45RO, and;
an antibody that specifically binds to CCR8.

36. A kit according to claim 34 or claim 35 wherein the antibodies are fluorescently labelled.
